(19) United States
(12) Patent Application Publication
(10) Pub. No.: US 2006/0269444 A1
(43) Pub. Date: Nov. 30, 2006

(54) Cardioscopy

(76) Inventors: Tomislav Mihaljevic, Beachwood, OH (US); Saeid R. Farivar, Brookline, MA (US); Lawrence H. Cohn, Brookline, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/595,042

(22) PCT Filed: Jul. 2, 2004
(86) PCT No.: PCT/US04/21247

§ 371 Date: Jun. 1, 2006

Related U.S. Application Data
(60) Provisional application No. 60/486,579, filed on Jul. 11, 2003.

(51) Int. Cl. A61M 37/00; A61M 1/00

(52) U.S. Cl. 422/45; 604/4.01; 604/6.14

(57) ABSTRACT

A method of cardioscopy may include creating a primary heart bypass circuit for perfusing an organism, creating a secondary circuit for perfusing the heart of the organism with a non-observation-impairing pumping medium, and observing the heart through the secondary circuit. A cardioscopy apparatus may include a primary heart bypass circuit for perfusing an organism, a secondary circuit for perfusing the heart of the organism with a non-observation-impairing pumping medium, and an observation device for observing the heart through the secondary circuit.

### BACKGROUND

Medical procedures in the heart are often performed by temporarily arresting the heart and diverting blood through an external cardiopulmonary bypass system. Although arresting the heart can be advantageous because the heart tissue is immobile and possibly easier to manipulate, the function of the heart (stroke volume, afterload, end diastolic volume, etc.) cannot be assessed. Furthermore, the anatomy of the immobilized heart may be distorted because the heart chambers are collapsed. The distorted anatomy can limit the value of intracardiac observation and can complicate intracardiac procedures; for example, a prosthetic device can be only approximately fit when the size and shape of relevant structures during beating is unknown.

### SUMMARY

Disclosed herein are systems and methods for cardioscopy during heart-lung bypass. Two circuits may be provided, one for perfusing an organism, and a second for perfusing the organism's heart. The heart may be observed, and/or an intracardiac procedure may be performed, through the second circuit.

In an embodiment, a method of cardioscopy can include creating a primary heart bypass circuit for perfusing an organism, creating a secondary circuit for perfusing the heart of the organism with a non-observation-impairing pumping medium, and observing the heart through the secondary circuit.

In an embodiment, a cardioscopy apparatus can include a primary heart bypass circuit for perfusing an organism, a secondary circuit for perfusing the heart of the organism with a non-observation-impairing pumping medium, and an observation device for observing the heart through the secondary circuit.

In an embodiment, the heart may be allowed to continue beating during practice of the method or use of the apparatus.

The pumping medium may have any of following exemplary characteristics, singly or in any combination: the pumping medium may be oxygenatable, oxygen-carrying, optically clear, translucent, and/or non-turbid. The pumping medium may include a perfluorocarbon, FLUOSOL® perfluorochemical emulsion, FLUORINERT® fluorinated organic composition, and/or other fluorocarbons.

In an embodiment, the observation device can be a visualization device, such as, for example, a camera, a light-sensitive system, an imaging modality, and/or other devices suitable for providing a visualization of an anatomical structure. The observation device can be an endoscope, angioscope, fiber-optic system, electrode, thermocouple, and/or other devices for providing an image, representation, assessment, or other characterization of an anatomical structure or region.

In an embodiment, a method can further include performing an intracardiac procedure. An apparatus can further include an intracardiac procedure device.

### BRIEF DESCRIPTION OF THE DRAWINGS

The drawings illustrate principles of the systems and methods disclosed herein and are not necessarily to scale. Implied absolute or relative dimensions are not limiting but are instead provided for illustrative purposes.

FIG. 1 schematically depicts a heart

FIG. 2 schematically depicts an exemplary primary bypass.

FIG. 3 schematically depicts an exemplary secondary circuit.

FIGS. 4-8 schematically depict exemplary alternative embodiments of a secondary circuit.

FIG. 9 schematically depicts an exemplary primary bypass and an exemplary secondary circuit.

FIG. 10 schematically depicts an exemplary observation device in the secondary circuit.

FIG. 11 schematically depicts an exemplary observation device and an exemplary instrument in the secondary circuit.

### DETAILED DESCRIPTION

The disclosed systems and methods facilitate the diagnostic and therapeutic manipulation of the heart by permitting observation of the heart through a dedicated circuit.

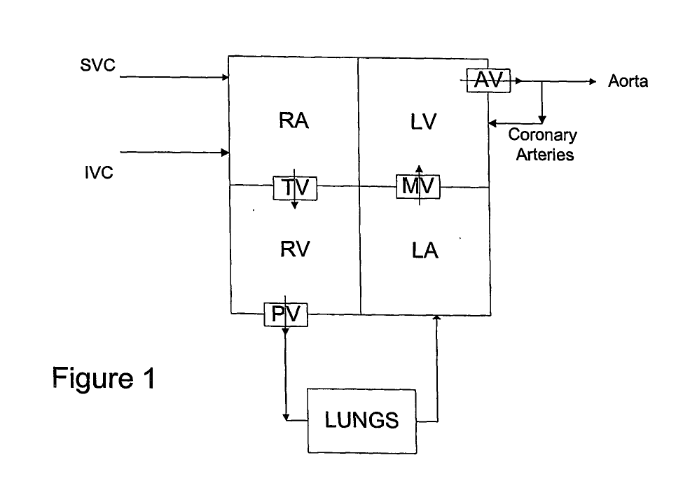

FIG. 1 schematically depicts a heart having four chambers: right atrium (RA), right ventricle (RV), left atrium (LA), and left ventricle (LV). The right atrium and right ventricle form the right heart, while the left atrium and left ventricle form the left heart. During normal blood flow, blood returning to the heart through the superior vena cava (SVC) and inferior vena cava (IVC) enters the right atrium. The blood is subsequently pumped through the tricuspid valve (TV) into the right ventricle and thence through the pulmonary valve (PV) and pulmonary arteries (PA) to the lungs. Blood returning from the lungs enters the left atrium, passes through the mitral valve (MV) into the left ventricle, and is finally pumped out of the heart, through the aortic valve (AV), and into the aorta. A portion of the ejected blood backflows into the coronary arteries, which perfuse the heart muscle itself.

The heart is susceptible to a variety of disease processes, such as myocardial ischemia, myocardial infarction, aneurysm, septal defects, valve incompetence, valve stenosis, and cardiomyopathies. Diagnostic evaluation and therapeutic intervention, such as biopsy, valve replacement, coronary angioplasty, may require myocardial arrest, or stoppage of the heart, so that surgical target structures are motionless. During such a procedure, oxygen must be provided to the remainder of the body. To achieve this, a primary bypass (“extracorporeal membrane oxygenation,” “heart-lung machine”) may be established that diverts blood from the heart, passes it through an oxygenator and a pump, and returns it to the arterial tree.

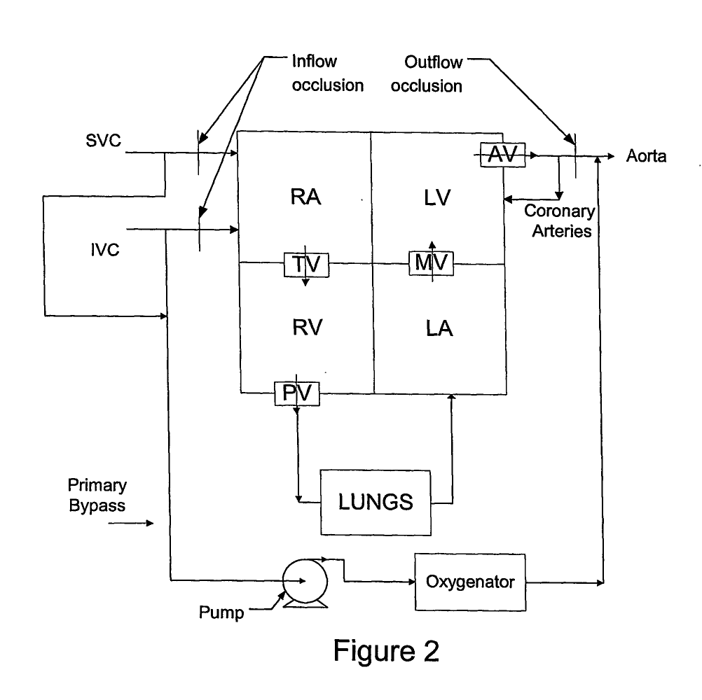

FIG. 2 schematically depicts an exemplary primary bypass. Inflow occlusions may be established in the SVC and IVC, and an outflow occlusion may be established in the aorta. An occlusion may be achieved, for example, with a clamp, a balloon, a space-filling catheter, and other techniques known to one of ordinary skill. Blood is drained from the SVC and IVC, e.g. by a drain catheter, directed through a pump, and passed through an oxygenator, which permits gas exchange for red blood cells and substitutes for the bypassed lungs. The heart may be arrested by, for example, cooling, infusion of a cardioplegic material such as potassium into the coronary vasculature, or other methods known to one of ordinary skill. The depicted embodiment is provided for exemplary purposes only; other components or arrangements are contemplated.

When the heart is bypassed and arrested in this manner, its function cannot be evaluated, nor can the efficacy of a therapy by readily assessed, because the muscle is not contracting, and because the geometry of the heart is distorted. Normally, the heart chambers are filled with blood; when the heart is bypassed, the chambers are emptied of blood and thus collapse, thereby distorting the cardiac geometry. In this distorted state, several functional and anatomical parameters cannot be assessed. For example, a weakness or aneurysm in the heart may not be apparent because there is no pressure on it to adopt its pathologic bulging shape. As another example, valve leaflets are out of position and therefore do not coapt as they normally would, so valve competence cannot be easily determined. Moreover, wall and/or valve leaflet motion cannot be assessed because the heart is motionless; even if the heart were allowed to beat, the absence of a pumping medium (e.g., blood) would so distort the chamber geometry as to hamper meaningful observation.

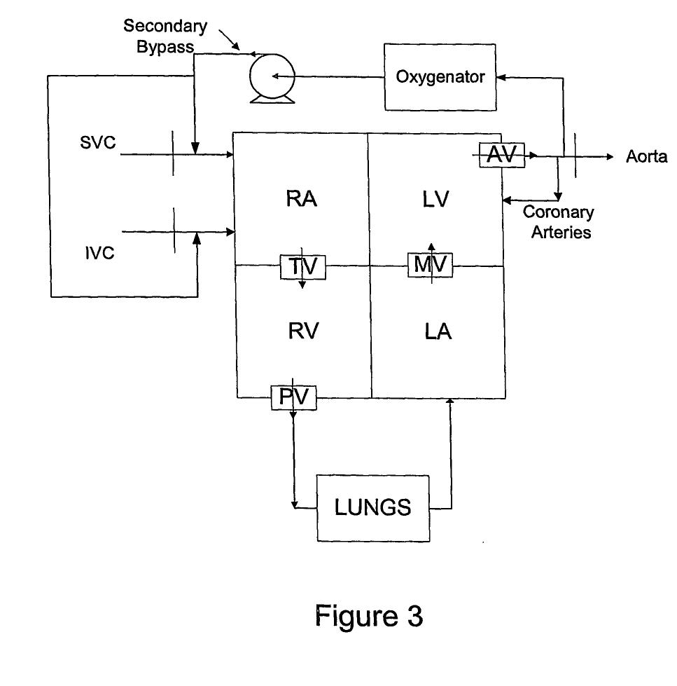

FIG. 3 schematically illustrates a secondary circuit which can be used to provide a pumping medium to a bypassed heart and facilitate observation of the heart therethrough. The second circuit may typically include the conduit, the pump (if provided), the oxygenator (if provided) and the portion of the heart and vasculature through which the pumping medium moves between the point of introduction and the point of collection. With SVC and IVC inflow occlusions and aortic outflow occlusions in place, a pumping medium (discussed below) may be introduced into the SVC and/or IVC. The medium may pass into the right atrium and flow through the heart and lungs in the conventional (antegrade) manner. Upon exiting the heart, the medium may be drawn off, e.g., by a drain catheter, directed through a pump and an oxygenator, and returned to the SVC and/or IVC. Owing to the aortic outflow occlusion, a portion of the ejected medium can backflow into the coronary arteries, thereby perfusing the myocardium with pump medium. The medium may be continuously pumped through the heart by receiving the medium from the aorta and returning the medium to the SVC and/or IVC. The outflow occlusion may also capture emboli which could have otherwise escaped into the systemic circulation.

The pump may be, for example, a centrifugal pump, a roller pump, a peristaltic pump, and/or one of a variety of other suitable pumps. The pump may include microfluidic components. The pump may be provided with a mechanism for matching the pump output to the cardiac output, to avoid under- or overfilling the heart, either of which can distort the cardiac anatomy. For example, cardiac output can be measured by conventional techniques prior to a procedure, and then the pump may be set to deliver the same output. Alternatively, as another example, cardiac output can be monitored during a procedure, as by a variety of methods, such as thermodilution, known in the art, and the pump adjusted to match the measured cardiac output. In an embodiment, the pump may be provided with a mechanism, such as a vent, to match pump output to the stroke volume of the heart. The secondary circuit may be fluidically isolated from the primary bypass, i.e., arranged so that pumping medium in the secondary circuit does not mix with material circulating in the primary bypass. Fluidic isolation may be provided by, for example, various outflow and/or inflow occlusions.

The pumping medium may include a wide variety of materials. The pumping medium may be non-observation-impairing, meaning that the physical properties of the medium do not prevent observation of the interior of the heart. In an embodiment, the pumping medium can be optically clear. The pumping medium can be transparent. The pumping medium can be translucent. The pumping medium can be non-turbid. In an embodiment, the pumping medium can be oxygenatable; e.g., capable of being loaded with oxygen. The pumping medium may be capable of delivering oxygen to tissue. In an embodiment, the pumping medium may be both oxygenatable and capable of delivering oxygen to tissue. Such a pumping medium can be used to perfuse the coronary vasculature of the heart. Examples of optically clear and/or otherwise non-observation-impairing fluids include flurocarbon-containing substances, such as FLUOSOL® and FLUORINERT® substances. The pumping medium may include perfluorocarbon emulsion. A variety of fluorocarbon compounds can take up oxygen and release oxygen to tissues.

The pumping medium may include other materials. The pumping medium may include blood. The pumping medium may include a material other than blood. The pumping medium may include blood treated with dimethylsulfoxide or other agents to lyse red blood cells. The pumping medium may include blood plasma. The pumping medium may include an anticoagulant. The pumping medium may include blood serum. The pumping medium may include cardioplegia, such as, for example, potassium cardioplegia.

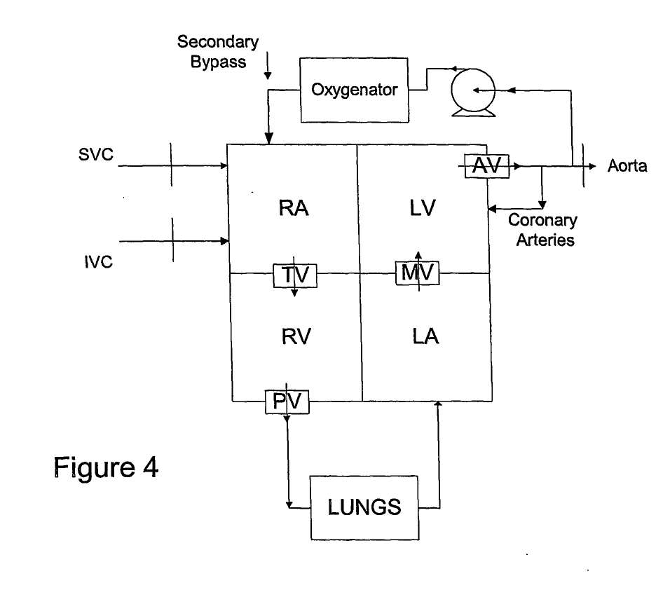

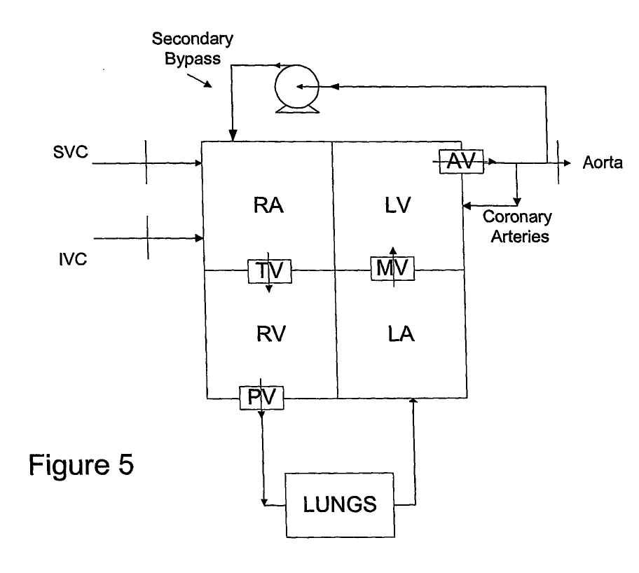

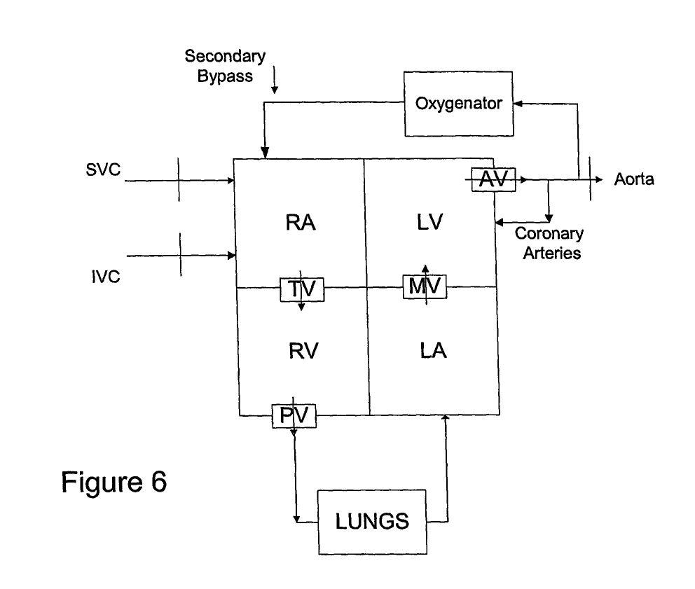

The secondary circuit can be established with a variety of configurations, examples of which are schematically depicted in FIGS. 4-8. As shown in FIG. 4, a secondary circuit can be established so that blood may be returned to the right atrium instead of to (or in addition to) the SVC and/or IVC. FIG. 5 depicts an embodiment in which the oxygenator is omitted from the secondary circuit. In this exemplary embodiment, the lungs may be relied upon to provide oxygenation. Alternatively, the oxygenator may be omitted in circumstances for which oxygenation is not required or is not desired, such as brief procedures or experimental procedures. In the exemplary embodiment shown in FIG. 6, the pump is omitted from the secondary circuit, and the heart may be relied upon to provide the motive force.

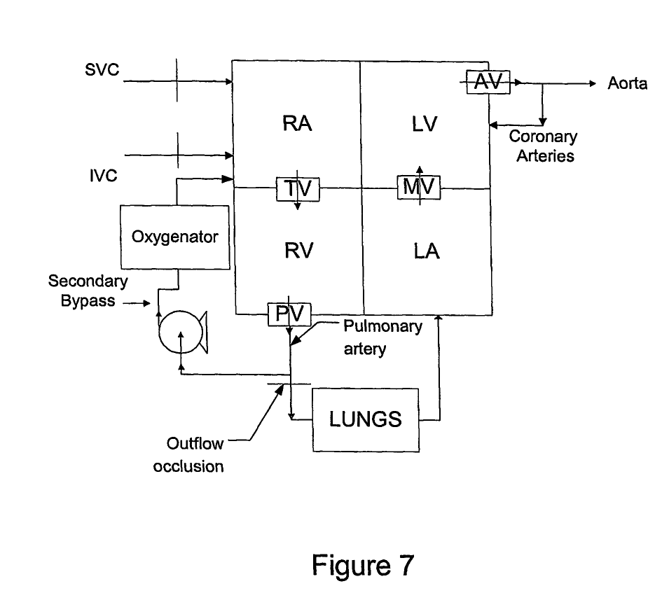

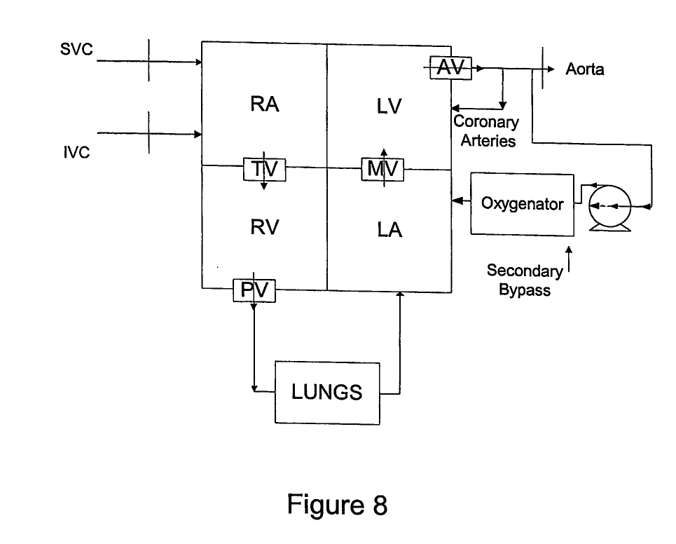

FIGS. 7-8 schematically depict embodiments in which only selected portions of the heart may be engaged by the secondary circuit. In the exemplary embodiment shown in FIG. 7, the right heart alone may be included in the secondary circuit. The pumping medium may be received from the pulmonary artery (and/or branches thereof) and returned to the right atrium. Such a circuit might be used, for example, when evaluation or treatment of the right heart to assess function of the tricuspid valve or the pulmonary valve, or in other situations for which left heart function is not needed or desired. As shown in FIG. 8, the secondary circuit may include the left heart alone. Pumping medium may be received from the aorta and reintroduced into the left atrium. Such a circuit might be used, for example, when evaluation or treatment of the left heart to assess function of the mitral valve or the aortic valve, the status of the left ventricular wall or interventricular septum, or in other situations for which right heart function is not needed or desired. A right heart secondary circuit and a left heart secondary circuit may be established concurrently to supply all four chambers of the heart while excluding the lungs. Accordingly, either half of the heart may be isolated from the other half of the heart for diagnostic and/or therapeutic purposes. In addition, a single chamber of the heart can be isolated from the other chambers.

In addition to facilitating anterograde flow through the heart, the disclosed systems and methods may also be practiced with retrograde flow. Retrograde flow may be performed, for example, by introducing pump medium into the aorta, receiving it from the SVC and/or IVC, and directing it through a pump. Analogous retrograde flow may be performed with other receive and return locations discussed above. For example, retrograde flow can be established through the heart and lungs. Retrograde flow can also be established through one or more chambers of the heart. Retrograde flow can involve flow through incompetent valves. Alternatively, valves may be stented open to facilitate retrograde flow through the chambers.

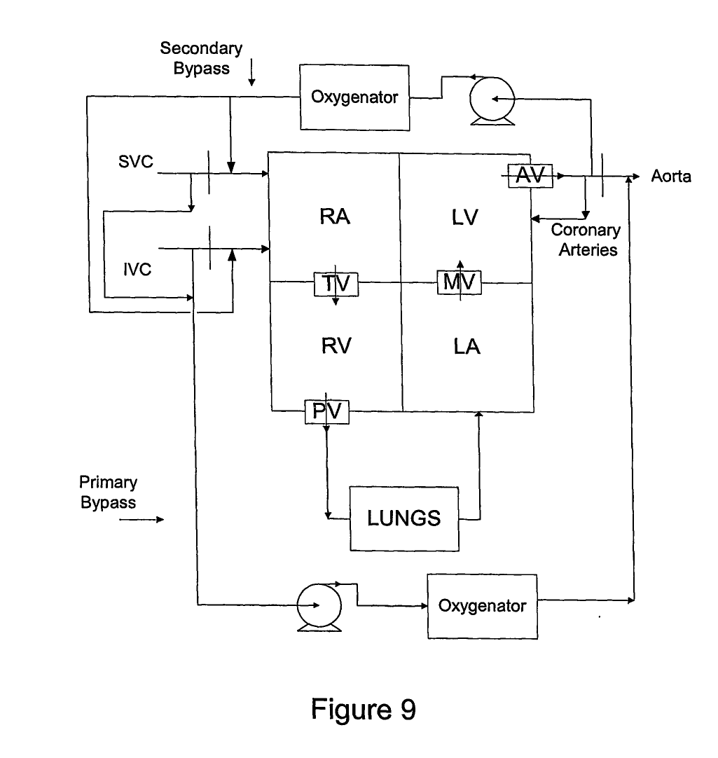

As shown in FIG. 9, a primary bypass may be established concurrently with a secondary circuit. FIG. 9 schematically depicts an exemplary embodiment in which both the primary bypass and the secondary circuit are established. The primary bypass may provide oxygenated blood to a subject, while the secondary circuit may provide pumping medium to one or more chambers or portions of the heart. In the depicted embodiment, the secondary circuit includes all four heart chambers and the lungs; however, one or more other secondary circuits may be established as well, such as the right heart circuit, the left-heart circuit, simultaneous right- and left heart circuits, circuits returning pumping medium to the right atrium, circuits returning pumping medium to the SVC and/or IVC, and the like.

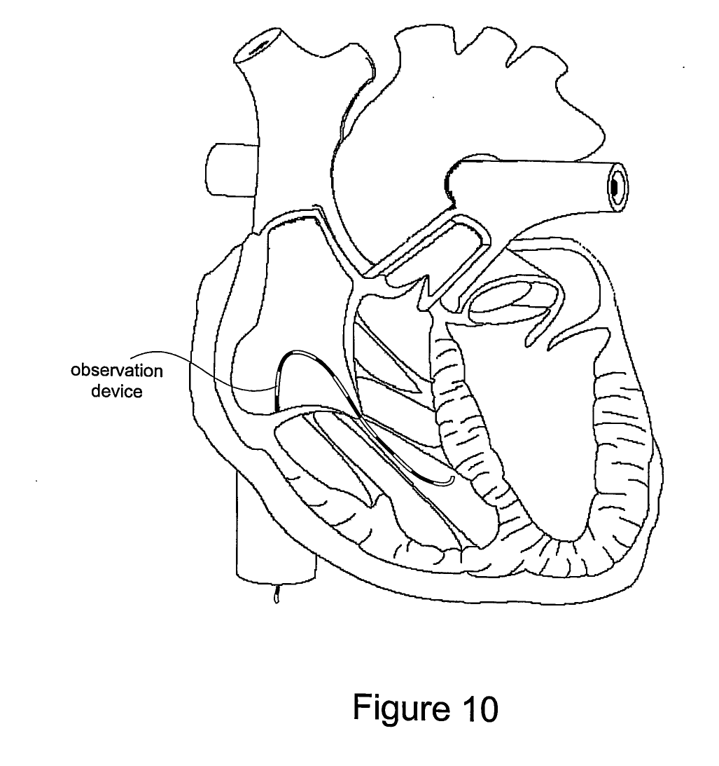

A non-observation-impairing pumping medium can facilitate observation of the heart. Observation may include a wide variety of modalities for determining information about an anatomic structure, including visualizing the anatomic structure, measuring a property of the structure, such as electrical potential, assessing uptake of a marker, such as a radioactive marker, and a wide variety of other modalities. Visualization may include systems for generating image data of an anatomic structure, such as optical imaging (for example, a fiber-optic system), CT, MRI, ultrasound, and other systems for obtaining images. Visualization may include an illumination source, such as a light source. FIG. 10 depicts an exemplary deployment of an observation device in the heart. The observation device may be introduced into the heart through the secondary circuit. For example, the observation device may be a catheter (such as an ultrasound catheter or an MRI catheter) or loaded on a guidewire and advanced through the device providing return flow to the heart, such as a lumen of an occlusion catheter. The observation device may be advanced through the lumen of an occlusion catheter, in apposition to the pumping medium. The observation device may be introduced anywhere along the secondary circuit and not necessarily only through the points of introduction and collection of the pumping medium. For example, while the pumping medium could be introduced into the secondary circuit via the IVC, the observation device could be introduced into the left atrium. In the exemplary depicted embodiment, a scope can be positioned to visualize the mitral valve.

Observation can be facilitated by using a non-observation-impairing pumping medium in the secondary circuit. For example, the pumping medium may be optically clear and the observation can be visualization, so that image quality is not degraded by a pumping medium that obscures the view of the heart. The selection of pumping medium may be guided by the degree of optical clarity or nonobservation impairment preferred for a particular procedure. For example, an optical visualization may benefit from use of an optically clear or translucent pumping medium, while other techniques might produce adequate results with use of more turbid medium that nevertheless does not impair observation using the selected observation technique. In an embodiment, a pumping medium that absorbs a particular wavelength may be rendered effectively optically clear by appropriately filtering an illumination source.

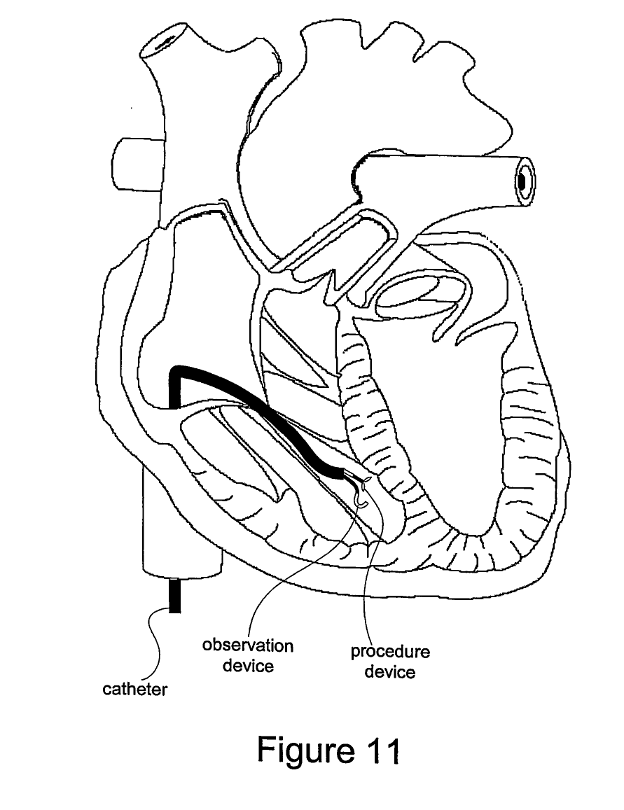

As shown in FIG. 11, use of a non-observation-impairing pumping medium can also facilitate diagnostic and/or therapeutic procedures in the heart. In the depicted exemplary embodiment, an observation device and a procedure device are provided through a catheter and are positioned to perform a therapy on the interventricular septum. An intracardiac procedure device may be introduced into the heart for performing a procedure on the heart. The procedure device may be introduced through the secondary circuit. The procedure device may be observed by the observation device. In an embodiment, the procedure device may be visualized by a camera of the observation device. Exemplary procedures include repair and/or closure of atrial and/or ventricular septal defects, biopsy of the heart, anti-arrhythmic ablation therapy, and valve repair and/or replacement.

What is claimed is:

1. A method of cardioscopy, comprising:
creating a primary heart bypass circuit for perfusing an organism;
creating a secondary circuit for perfusing the heart of the organism with a non-observation-impairing pumping medium; and
observing the heart through the secondary circuit.

2. The method of claim 1, further comprising allowing the heart to continue beating.

3. The method of claim 1, wherein the pumping medium is oxygenatable.

4. The method of claim 1, wherein the organism is perfused with blood.

5. The method of claim 1, wherein creating the primary heart bypass circuit includes receiving blood from a vena cava and returning blood to the aorta.

6. The method of claim 1, wherein creating the primary heart bypass circuit includes perfusing a coronary blood vessel.

7. The method of claim 1, wherein the secondary circuit is fluidically isolated from the primary heart bypass circuit.

8. The method of claim 1, wherein creating the second circuit includes continuously perfusing the heart.

9. The method of claim 1, wherein creating the secondary circuit includes perfusing a chamber of the heart.

10. The method of claim 1, wherein creating the secondary circuit includes perfusing a coronary blood vessel.

11. The method of claim 1, wherein creating the secondary circuit includes receiving the pumping medium from the aorta and returning the pumping medium to a vena cava.

12. The method of claim 1, wherein the pumping medium is optically clear.

13. The method of claim 12, wherein the optically clear pumping medium is oxygenatable.

14. The method of claim 1, wherein the pumping medium is translucent.

15. The method of claim 1, wherein the pumping medium is non-turbid.

16. The method of claim 1, wherein the pumping medium includes a fluorocarbon.

17. The method of claim 16, wherein the fluorocarbon is perflurocarbon.

18. The method of claim 1, wherein observing includes visualizing the heart through a catheter.

19. The method of claim 1, wherein observing includes visualizing with an angioscope.

20. The method of claim 1, wherein observing includes visualizing with an endoscope.

21. The method of claim 1, wherein observing includes observing a heart chamber.

22. The method of claim 1, wherein observing includes observing a heart valve.

23. The method of claim 1, further comprising performing an intracardiac procedure.

24. A cardioscopy apparatus, comprising:
a primary heart bypass circuit for perfusing an organism;
a secondary circuit for perfusing the heart of the organism with a non-observation-impairing pumping medium; and
an observation device for observing the heart through the secondary circuit.

25. The apparatus of claim 24, wherein the pumping medium is oxygenatable.

26. The apparatus of claim 24, wherein the organism is perfused with blood.

27. The apparatus of claim 24, wherein the primary heart bypass circuit receives blood from a vena cava and returns blood to the aorta.

28. The apparatus of claim 24, wherein the primary heart bypass circuit perfuses a coronary blood vessel.

29. The apparatus of claim 24, wherein the secondary circuit is fluidically isolated from the primary heart bypass circuit.

30. The apparatus of claim 24, wherein the second circuit continuously perfuses the heart.

31. The apparatus of claim 24, wherein the secondary circuit perfuses a chamber of the heart.

32. The apparatus of claim 24, wherein the secondary circuit perfuses a coronary blood vessel.

33. The apparatus of claim 24, wherein the secondary circuit receives the pumping medium from the aorta and returns the pumping medium to a vena cava.

34. The apparatus of claim 24, wherein the pumping medium is optically clear.

35. The apparatus of claim 34, wherein the optically clear pumping medium is oxygenatable.

36. The apparatus of claim 24, wherein the pumping medium is translucent.

37. The apparatus of claim 24, wherein the pumping medium is non-turbid.

38. The apparatus of claim 24, wherein the pumping medium includes a fluorocarbon.

39. The apparatus of claim 38, wherein the fluorocarbon is perfluorocarbon.

40. The apparatus of claim 24, wherein the observation device comprises an intracardiac visualization device.

41. The apparatus of claim 24, wherein the observation device comprises a catheter.

42. The apparatus of claim 24, wherein the observation device comprises an angioscope.

43. The apparatus of claim 24, wherein the observation device comprises an endoscope.

44. The apparatus of claim 24, further comprising an intracardiac procedure device.
